(19) United States
(12) Patent Application Publication
(10) Pub. No.: US 2022/0226107 A1
(43) Pub. Date: Jul. 21, 2022

(54) EMBOLIC PROTECTION ACCESS SYSTEM

(71) Applicant: ENCOMPASS TECHNOLOGIES, INC., San Clemente, CA (US)

(72) Inventors: Michael Jones, San Clemente, CA (US); George Wallace, San Clemente, CA (US)

(21) Appl. No.: 17/657,795

(22) Filed: Apr. 4, 2022

Related U.S. Application Data
(62) Division of application No. 16/868,076, filed on May 6, 2020.
(60) Provisional application No. 62/888,897, filed on Aug. 19, 2019.

(51) Int. Cl. A61F 2/24; A61M 25/00; A61M 25/01; A61F 2/01

(52) U.S. Cl. CPC A61F 2/243; A61M 25/003; A61M 25/0108; A61F 2/011; A61F 2002/016; A61M 2205/7545; A61M 2025/0024; A61M 2210/127; A61F 2230/0069; A61F 2/013

(57) ABSTRACT

Methods and devices are provided for protecting the cerebrovascular circulation from embolic debris released during an index procedure. An embolic protection filter is delivered in a reduced profile configuration via an access catheter, and positioned in the aorta spanning the ostia to the three great vessels leading to the cerebral circulation. An index procedure catheter is thereafter advanced through the same access catheter to conduct the index procedure. The index procedure may be a transcatheter aortic valve replacement.

### CROSS-REFERENCE TO RELATED APPLICATIONS

This application is a divisional application of U.S. patent application Ser. No. 16/868,076 filed May 6, 2020, which claims the priority benefit under 35 U.S.C. § 119(e) of U.S. Provisional Application No. 62/888,897, filed Aug. 19, 2019, the entirety of each of which is hereby incorporated by reference herein.

### BACKGROUND OF THE INVENTION

### Field of the Invention

The present invention relates to protection of one or more side branch vessels from a parent vessel, such as for protection of the cerebral vasculature during a surgical or interventional procedure of the type that might dislodge embolic debris.

### Description of the Related Art

There are four arteries that carry oxygenated blood to the brain, i.e., the right and left vertebral arteries, and the right and left common carotid arteries. The right vertebral and right common carotid are both supplied via the brachiocephalic artery. Thus at the aortic arch the cerebral circulation is supplied via the brachiocephalic, the left common carotid and left subclavian arteries.

Various procedures conducted on the human body, e.g., transcatheter aortic valve replacement (TAVR), aortic valve valvuloplasty, carotid artery stenting, closure of the left atrial appendage, mitral valve annuloplasty, repair or replacement, can cause and/or dislodge materials (whether native or foreign), these dislodged bodies can travel into one or more of the arteries supplying the brain resulting in, inter alia, stroke. Moreover, atheromas along and within the aorta and aortic arch can be dislodged as the TAVR catheter is advanced toward the diseased aortic valve and subsequently withdrawn after implantation is completed. In addition, pieces of the catheter itself can be stripped away during delivery and implantation. These various forms of vascular debris, whether native or foreign, can then travel into one or more cerebral arteries, embolize and cause, inter alia, a stroke or strokes.

Intraoperative embolic stroke is one of the most significant complications of cardiac, aortic and vascular procedures, diagnosed in 1-22% of patients undergoing cardiovascular surgery. Even more frequently, in up to 70% of cases, patients undergoing heart, valve, coronary artery bypass and aortic surgery experience subclinical embolic events as recorded by transcranial Doppler and MRI. Recent data showed an astounding incidence of stroke as detected by MRI in practically all groups of cardiac patients: in TAVR v—84%, Aortic Valve Replacement—52%, emergent coronary intervention—49%, Balloon Aortic Valvuloplasty—40%, Cardiac Ablation 38% and Coronary Artery Bypass Surgery—20%. These embolic events lead to cognitive impairment and disability and have a significant impact on patients' recovery.

The main sources of cerebral emboli and stroke in this setting resides in the heart, heart valves, thoracic aorta, and great vessels when these structures are invaded. Even simple cardiac catheterization with an endovascular catheter can induce trauma of the atherosclerotic thoracic aorta leading to formation of embolic particles with subsequent embolic brain injury ranging from latent ischemic foci to a massive or even fatal stroke.

A variety of devices have been proposed that attempt to prevent embolization of the carotid arteries during endovascular and cardiac interventions. These anti-embolic devices, however, have not received wide acceptance due to their complexity and invasive character with the risk of additional trauma to the inner vessel wall resulting in a high risk to benefit ratio. Known devices require insertion of additional hardware into the arterial system or aorta, a procedure that is known by itself to be associated with all classical risks of endovascular intervention, and also multiple catheters risk mechanical entanglement or additional remote vascular access sites.

### SUMMARY OF THE INVENTION

There is provided in accordance with one aspect of the present invention, a method of protecting the cerebral vascular circulation from embolic debris released during an index procedure. The method comprises providing an embolic protection delivery catheter having a tubular embolic protection filter in a reduced profile configuration, the filter having a self expandable wire frame, a filter membrane carried by the frame and a proximal and distal radiopaque markers. The embolic protection delivery catheter is advanced through an access sheath or catheter such as a TAVR procedural access catheter to position the distal marker on an upstream side of a side vessel and the proximal marker on a downstream side of a side vessel in the aorta. The embolic protection delivery catheter is proximally retracted to expose the filter and permitting the frame to radially expand, spanning at least one and preferably three side vessels. An index procedure catheter is thereafter advanced through the same access catheter to conduct the index procedure.

A control wire may be provided, extending proximally from the filter and through the sheath, alongside of the index procedure catheter. The index procedure may comprise a TAVR.

The distal marker may be positioned on an upstream side of the brachiocephalic artery, and the proximal marker may be positioned on a downstream side of the left subclavian artery.

The method may additionally comprise the step of retracting a suture along side or through the control wire to reduce the diameter of the proximal end of the filter, to facilitate retraction of the filter back inside of the embolic protection delivery catheter, following completion of the index procedure.

In accordance with another aspect of the invention, there is provided an embolic protection access system. The system comprises a self expandable frame having a proximal end and a distal end; a filter membrane supported by the frame; a bare metal leading segment extending distally (upstream) beyond the filter membrane; and a tubular control wire extending proximally from the frame. The frame may be tubular with an arcuate filter membrane that extends less than a full circumference of the frame, or the filter membrane may also be tubular. The frame may comprise woven wire filaments or laser cut tube stock to provide a plurality of interconnected struts separated by side wall openings. The frame may be balloon expandable but is preferably self expandable or both and able to conform to the anatomy at the deployment site.

The proximal end of the frame may reside on a plane that extends at a non-normal angle (e.g. less than 90°) to a longitudinal axis of the tubular frame, to present an inclined proximal face to facilitate recapture. The proximal end of the frame may include a plurality of eyelets. An eyelet may be formed by an apex at the junction of two struts of a wire filament. A suture may extend through the eyelets and be configured to collapse the proximal end of the filter upon proximal retraction of the suture.

The control wire may comprise a central lumen, and the suture may extend axially through the central lumen. The embolic protection access system may further comprise a tubular delivery catheter, and the tubular frame may be carried in a reduced cross-sectional configuration within the delivery catheter. The delivery catheter may have an outer diameter of less than the ID of the TAVR sheath, such as no more than about 13.9 F, and generally within the range of from about 6-13.9 F. In one implementation, the OD of the delivery catheter is about 13.5 F.

### BRIEF DESCRIPTION OF THE DRAWINGS

FIG. 1 illustrates an access catheter positioned in the descending aorta with a guide wire extending across the aortic arch and through the aortic valve.

FIG. 2X is a cross-section taken along the lines X-X in FIG. 1.

FIG. 3 is a side elevational schematic cross-section through the distal end of an embolic protection access system.

FIG. 4 is a schematic view of the embolic protection system constrained within a deployment catheter and positioned across the aortic arch.

FIG. 5X is a cross-sectional view taken along the lines X-X of FIG. 1, at the procedural stage illustrated in FIG. 4.

FIG. 6 is a schematic view of the embolic protection access system filter deployed across the aortic arch.

FIG. 7 illustrates a trans catheter aortic valve replacement catheter.

FIG. 8 illustrates the trans catheter aortic valve replacement catheter deploying an aortic valve through the embolic protection access sheath of the present invention.

FIG. 9X is a cross-sectional view taken along the lines X-X of FIG. 1, at the procedural stage illustrated in FIG. 8.

FIG. 10 illustrates retrieval of the embolic projection access system filter.

FIG. 11 is a schematic view of an embolic protection access system filter.

### DETAILED DESCRIPTION OF THE PREFERRED EMBODIMENT

The protective sheath of the present invention is designed to provide vascular protection and filtering of debris that can be created during interventional procedures. In one exemplary use, the sheath will protect the aortic arch during the passage of interventional devices whose destination is the heart. The protective sheath will preferably cover all three great vessels (brachiocephalic, left common carotid and left subclarian blood vessels) leading to the brain. Filtering and/or deflecting debris that would otherwise go to the brain will protect against a stroke and other negative impact to cognitive functions.

Trans-catheter Aortic Valve Replacement (TAVR) for example, is a popular and growing interventional cath lab catheter procedure that creates debris capable of causing a stroke, or other cerebral complications. Although embolic protection systems have been proposed in the past, such systems generally require an additional vascular access point and/or additional catheter exchange steps. The protective sheath of the present invention can be placed directly through the procedural sheath obviating the need for a separate access site.

Vascular access via the femoral artery can be accomplished, for example, using a Perclose ProGlide system (Abbott Vascular) as is understood in the art. This places one or two sutures in the femoral artery at the start of the procedure. These can be used to close 14 F or larger puncture sites in the groin at the end of the procedure. A hollow needle is first introduced from the groin into the femoral artery. A guidewire is introduced through the needle and into the blood vessel. The needle is withdrawn and a blunt cannula with a larger outside sheath is placed over the wire and advanced into the artery. The blunt cannula can then be withdrawn, leaving the access sheath positioned typically in the descending aorta, above the renal arteries, where it is available for various procedural catheters and guidewires to be introduced and exchanged through the access sheath.

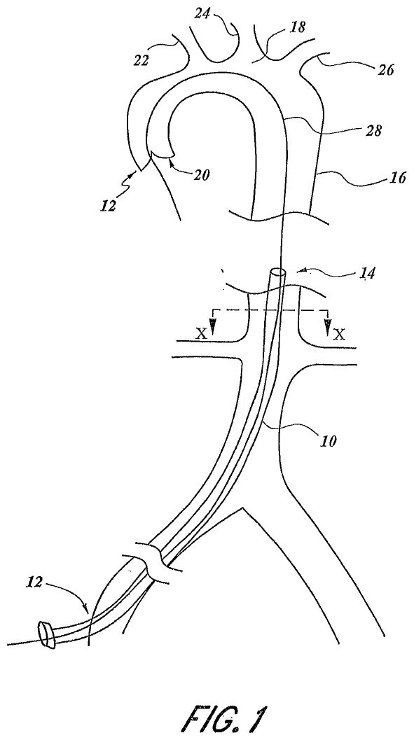

FIG. 1 illustrates an access sheath 10 extending from a femoral artery access point 12 to position a distal end 14 of the access sheath 10 in the descending aortic artery 16 and available to guide a guidewire 28 and procedure catheters superiorly such as to the aortic arch 18 or the aortic valve 20 or beyond into the heart. The initial access needle and blunt cannula have been removed. In the specific procedure described primarily herein, the access sheath is available to guide devices of the present invention to regulate the flow of embolic debris through the ostia of the brachiocephalic artery 22, the left common carotid artery 24 and the left subclavian artery 26 thereby protecting the cerebral circulation.

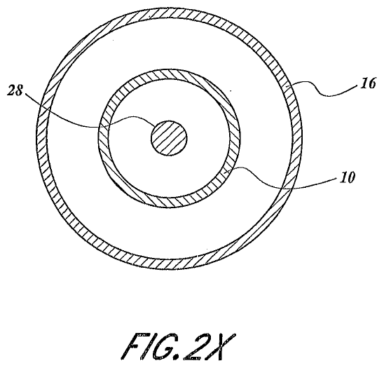

A suprarenal cross section through the aorta along the lines X-X on FIG. 1 is shown in FIG. 2X, in which the blunt cannula has been removed and a guidewire 28 extends through the expandable TAVR access sheath 10 which may have an ID, for example, of no more than about 28 F or 20 F or no more than about 15 F and in one implementation an ID of about 14 F depending primarily upon the size of the TAVR delivery catheter index procedure catheter size.

The guidewire 28, such as an 0.035″ guidewire, is advanced through the aorta over the arch 18 through the aortic valve 20 (see FIG. 1), and into the ventricle (not illustrated). Preferably an exchange length (e.g., 260 cm or longer) guidewire is used to facilitate catheter exchanges.

The 14 French ID TAVR procedural sheath 10 (18.5 F outside diameter, 22 F expanded outside diameter) is advanced over the 0.035″ guidewire beyond the renal arteries and into the descending aorta 16. This procedure sheath is the same sheath that provides access for the catheter 30 of the embolic protection system of the present invention.

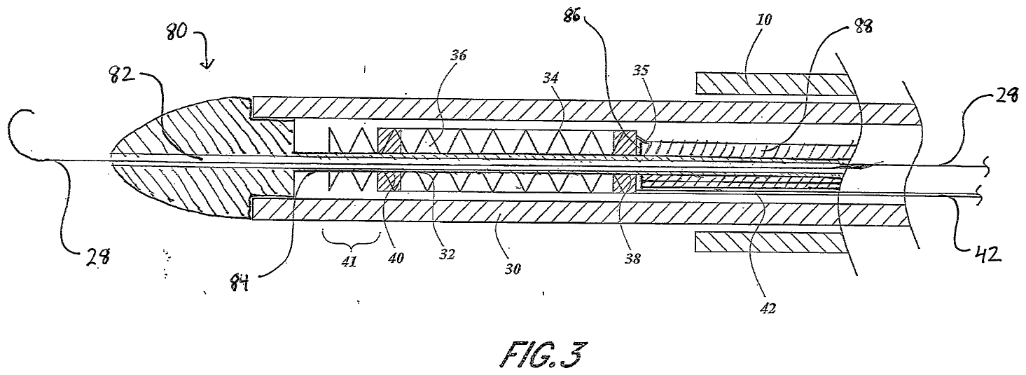

Referring to FIG. 3, there is illustrated an Embolic Protection and Access and delivery (EPA) catheter 30 having, for example, a 13.5 F tubular body, advanced through the 14 F TAVR delivery sheath 10. The frame and filter are back loaded into the EPA catheter 30 prior to advancing the catheter 30 over the guidewire 28 and through the access sheath 10. The EPA catheter 30 is thereafter axially advanceable beyond the distal end of the 14 F delivery sheath 10.

EPA catheter 30 additionally comprises a distal nose cap 80 axially distally displaceable from the distal end of the tubular side wall of catheter 30. Distal nose cap 80 includes an atraumatic distal tip, and a central lumen 82 for movably receiving guide wire 28. Nose cap 80 is carried by an inner support tube 84 which extends proximally to a distal end face 86 of a push tube 88 which extends to a push tube control on or associated with the proximal manifold (not illustrated). Tubular support tube 84 includes a central lumen 82, for slidably receiving guide wire 28 there through. The OD of inner support tube 84 is less than the OD of pusher tube 88, creating an annular distal end face 86 to prevent proximal movement of the expandable frame 34. Proximal retraction of the tubular body of catheter 30 with respect to the pusher 88 exposes the filter 32 which can radially expand into position across the aortic arch.

A one or two or preferably three vessel filter 32 is positioned in a collapsed configuration within the 13.5 or 13.9 F EPA catheter 30. The filter 32 comprises an expandable frame 34 which carries a filter membrane 36 over at least a portion thereof. See also FIG. 11. In the illustrated embodiment, the filter membrane 36 is carried by the frame 34 from a proximal marker 38 to a distal marker 40 which mark the ends of the filter cover. Additional markers may be desirable to mark the ends of the frame (such as the distal end which extends beyond the filter membrane) in the event that the frame struts are not easily seen under fluoroscopic imaging. The frame distally of the distal marker 40 is an uncovered landing zone 41 with bare metal struts or may have a coating but has open sidewall windows between adjacent struts without the membrane 36.

The membrane may be configured to block the passage of debris as small as 0.5 mm and greater, or 0.25 mm and greater, or 0.1 mm and greater or less. The membrane may be formed by an electrospinning process. Electrospinning refers generally to processes involving the expulsion of flowable material from one or more orifices, and the material forming fibers are subsequently deposited on a collector. Examples of flowable materials include dispersions, solutions, suspensions, liquids, molten or semi-molten material, and other fluid or semi-fluid materials. In some instances, the rotational spinning processes are completed in the absence of an electric field. For example, electrospinning can include loading a polymer solution or dispersion, including any of the cover materials described herein, into a cup or spinneret configured with orifices on the outside circumference of the spinneret. The spinneret is then rotated, causing (through a combination of centrifugal and hydrostatic forces, for example) the flowable material to be expelled from the orifices. The material may then form a “jet” or “stream” extending from the orifice, with drag forces tending to cause the stream of material to elongate into a small diameter fiber. The fibers may then be deposited on a collection apparatus. Further information regarding electrospinning can be found in U.S. Publication No. 2013/0190856, filed Mar. 13, 2013, and U.S. Publication No. 2013/0184810, filed Jan. 15, 2013, which are hereby incorporated by reference in their entirety.

A control wire 42 extends from the frame 34 proximally to the proximal end of the catheter. Proximal motion of the catheter 30 relative to the control wire 42 will retract the catheter 30 to uncover the three vessel filter 32 leaving it unconstrained. This allows the frame 34 to self expand into, for example, a tubular configuration, having a diameter of at least about 20 mm or 25 mm to about 30 mm or 35 mm or more, and to support the membrane 36 against the wall of the aorta spanning the aortic arch and cover the three great vessels. Thus, the device can have an operating range of vessels having a diameter of from about 20 mm to about 35 mm. The unconstrained transverse cross sectional configuration can be less than a full annular side wall, such as a arcuate configuration extending no more than about 270° or 180° or less but having an arc length sufficient to span the ostia of the great vessels.

The filter 32 may be loaded into a collapsed configuration within the 13.5 French EPA catheter 30 by back loading the control wire 42 through the distal tip of the 13.5 F EPA catheter 30. The control wire 42 is proximally retracted, pulling the covered frame 34 into the tip of the EPA catheter 30. One or two or more ramped struts 35 or a purse string loop (discussed below) may be utilized to facilitate entry of the filter into the distal end of the EPA catheter 30. The 13.5 F EPA catheter 30 may then be loaded over the 0.035″ guidewire, into the 14 F ID sheath 10 and advanced distally into the blood vessel.

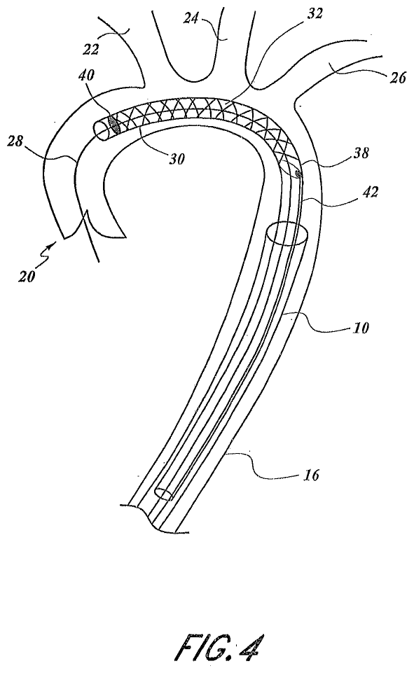

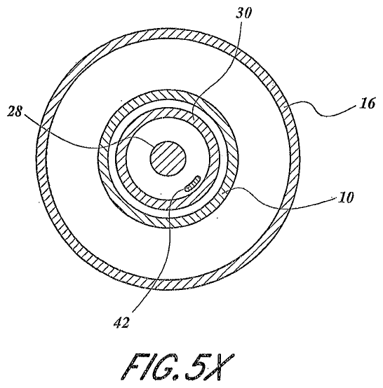

Referring to FIG. 4, the 13.5 F EPA catheter 30 with the covered frame is advanced distally with the collapsed filter 32 inside, until the ostia of the three great vessels are located in between the distal marker 40 and the proximal marker 38. The suprarenal cross section through the aorta along the lines X-X on FIG. 1 is shown in FIG. 5X as it may appear in this stage of the procedure, in which the EPA catheter 30 extends through and beyond guide catheter 10 and contains control wire 42 which leads distally to the three vessel filter positioned in the aortic arch.

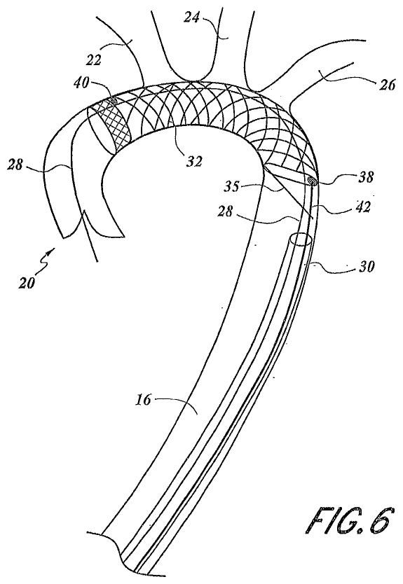

Referring to FIG. 6, once the markers 38 and 40 are confirmed to be on either side of the great vessels covering the aortic arch, the EPA catheter 30 is proximally retracted relative to the filter 32 to expose the uncovered distal landing zone 41 of the frame 34 so that it might radially expand and engage the wall of the aorta. The 13.5 F delivery catheter 30 may then be proximally withdrawn and removed from the patient. As the catheter 30 is retracted to expose the filter 32, the frame 34 will radially expand to cover at least the ostia along the aortic arch.

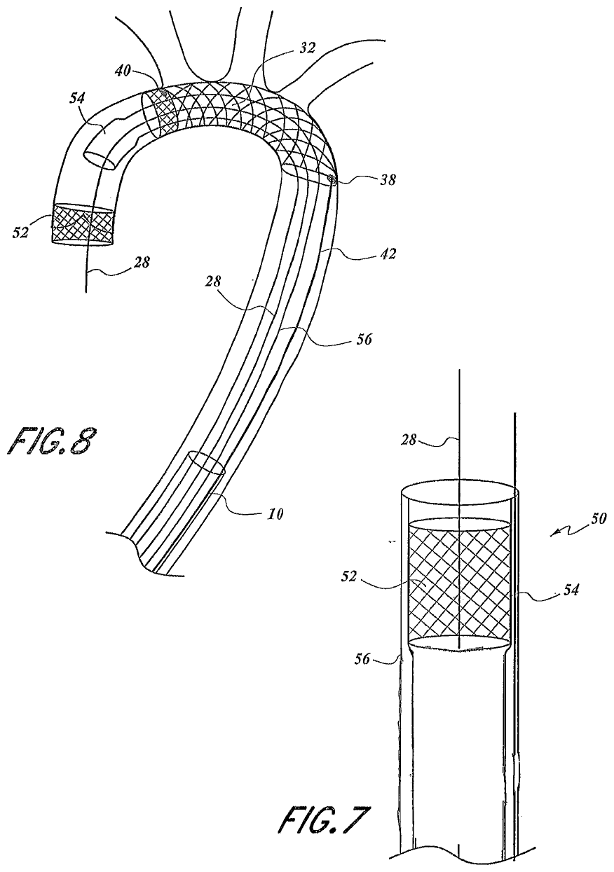

The basic construction of a TAVR delivery system 50 is shown in FIG. 7. A compressed valve and valve support frame 52 is carried within an expandable 14 F ID TAVR procedural delivery sheath 56. A valve pusher 54 is provided to deploy the valve 52. The loaded delivery system 50 is configured to advance over the guidewire 28.

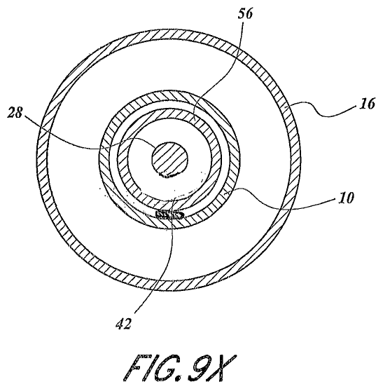

Referring to FIG. 8, the 13.5 F EPA delivery catheter 30 is proximally retracted over the 0.035″ guidewire 28 leaving the exchange guidewire 28 in place. The TAVR valve 52 with retention jacket and TAVR delivery pusher tube 54 both residing within the TAVR delivery catheter 56 are distally advanced over the 0.035″ guidewire to the desired valve (TAVR) deployment location. The TAVR valve is deployed and the TAVR delivery catheter 56 and pusher tube 54 are both withdrawn proximally from the body.

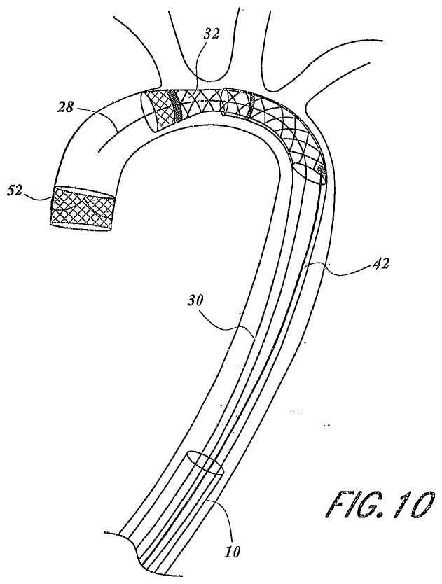

FIG. 9X shows the cross-sectional view taken along the line X-X of FIG. 1, at the stage of the procedure illustrated in FIG. 8. The TAVR delivery catheter 56 for delivering the TAVR valve 52 extends axially through and beyond the TAVR procedural sheath 10. The control wire 42 extends axially within the delivery sheath 10 and outside the and outside the TAVR delivery catheter 56.

Thus the delivery catheter 56 has replaced the EPA catheter 30 which has been removed, and the filter remains tethered by the flat control wire 42. Thus, the embolic protection system can be introduced via the same procedural sheath as is the TAVR valve, although it can also be introduced via a separate access site if desired.

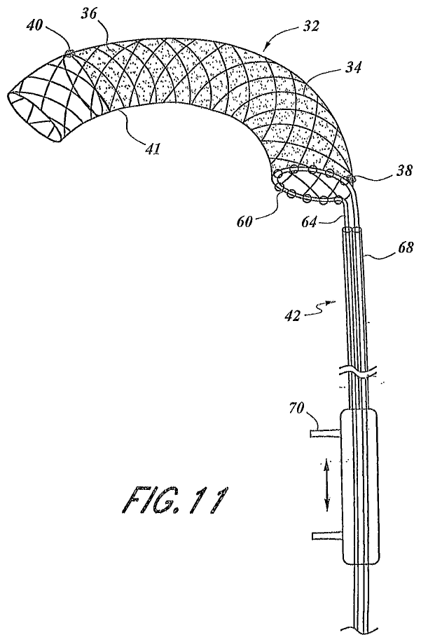

The embolic protection system may then be removed in the same procedure, or in a separate, subsequent procedure. Referring to FIG. 10, the 13.5 F EPA catheter 30 is advanced back over the 0.035″ guidewire and over the control wire 42. The 13.5 F catheter is distally advanced over the filter 32 while proximal traction is maintained on the control wire 42, to capture the covered frame and any trapped debris. The delivery system with recaptured filter may then be proximally retracted with or over the 0.035″ guidewire and withdrawn from the patient.

Additional details of the embolic protection system are shown in FIG. 11. The expandable frame 34 comprises a plurality of filaments joined at a plurality of apexes 60 surrounding the proximal opening to the tubular three vessel filter 32. A suture 62 may be threaded through the apexes 60 into a loop with at least one suture tail 64 extending proximally to a proximal manifold or control outside of the patient. Proximal retraction of the suture tail 64 with respect to the expandable frame 34 will cause the proximal opening of the tubular filter 32 to reduce in size, with a ‘purse string’ tightening effect. In the illustrated embodiment, the suture loops around the proximal opening to the filter, and produces a first suture tail 64 and a second suture tail 66 which extend all the way to the proximal end of the catheter.

The control wire 42 in this implementation is tubular having one or two lumens, and the suture tails 64 and 66 extend proximally through the central lumen or lumens of the control wire 42. Preferably, the tubular control wire 42 is flat (rectangular or oval in transverse cross section) or otherwise provided with a major axis in a circumferential direction that is greater than a minor axis in the radial direction when measured in cross-section. This allows the minimization of the space between the outside diameter of valve delivery catheter 56 and the inside diameter of the access sheath 10, as may be understood in connection with FIG. 9X.

The flat tube may be a tube with 2 lumens side by side and constructed as an extruded polymer, or as two metal tubes brazed or welded together along their length. It could alternatively be a round tube, which has slightly higher profile, depending upon the particular system. A round tube of about 0.030 inch or less will generally not have much negative impact on deploying the valve thru the introducer.

Alternatively, two wires may extend through the deployment catheter using the deployment catheter as the base of the noose to tighten and constrict the proximal end of the stent.

A single relatively large wire greater than about 0.010 inch diameter, may be used within the deployment catheter and be sufficiently controllable when left within the introducer sheath and aorta. Smaller wires (e.g., 0.010 or smaller) preferably extend through support tubes or tube control them and keep from tangling or getting in the way. The smaller wires make cinching the purse string easier due to the bend in small radii needed to close the purse string, but small wires need the support along their length to push out and release the cinch and open the proximal end of the stent.

An alternative is to provide a tube running from the handle to the stent and physically/permanently connected to the proximal end of the stent. A single wire has a distal end anchored to the frame adjacent the tube and extends around the circumference and through the braid tips and then passing proximally within the tube to the handle. This enables the pull/push on only a single wire to close/open the purse string.

To retrieve the filter 32 following completion of the index procedure, one or both suture tails 64, 66 are proximally retracted by manipulating a control such as by retraction of a slider switch 70 on the proximal handle. The distal end of the control wire 42 abuts and prevents proximal movement of the frame. Retraction of the suture thereby reduces the diameter of the proximal opening on the filter. That, along with the angled proximal face of the frame 34 allows the EPA catheter 30 to be distally advanced relative to the filter, to recapture the filter for removal as illustrated in FIG. 10.

The foregoing discussion has primarily been directed to positioning a filtration device in the aorta to provide cerebral protection during TAVR procedures, where during the catheter based procedure, debris from the Atrium, Aortic Valve, or Aorta can be dislodged, travel to the Aortic Arch 18 and enter the cerebral circulation through the great vessels (3) leading to the brain. However, the devices of the present invention can be utilized in any of a variety of peripheral, coronary or neurovascular environments where filtering or deflecting debris from entering a branch vessel off a parent vessel may be desired.

The cerebral protection system of the present invention may also be utilized during a variety of additional cardiovascular interventions where debris could be generated from the Left Ventricle, Mitral Valve, Left Atrium, Aortic Valve, or Aorta and enter the great vessels (3) to the brain. These include other valvular surgery procedures such as open aortic valve replacement, open mitral valve replacement, open mitral valve repair, trans-catheter mitral Valve Replacement (TMVR), and balloon valvuloplasty. Additional index procedures include, circulatory support such as with the Impella pump, Left Ventricular assist devices, Electro Physiology Ablation (A-Fib), Left Atrial Appendage closure, Atrial Septal Defects (ASD), PFO closure procedures, and other cardiac surgery where bypass is utilized.

Any procedure that is performed with access from the arterial side would allow the embolic protection device and procedure of the present invention to be performed through the procedural access sheath. Procedures that require open access, or venous access would require a separate access site.

What is claimed is:

1. A method of protecting the cerebro vasculature from embolic debris, comprising:
providing an embolic protection delivery catheter having a tubular embolic protection filter in a reduced profile configuration, the filter having a self expandable wire frame, a filter membrane carried by the frame and proximal and distal radiopaque markers;
trans vascularly advancing the embolic protection delivery catheter through an access sheath to position the distal marker on an upstream side of a side vessel and the proximal marker on a downstream side of a side vessel in the aorta;
retracting the embolic protection delivery catheter to expose the filter and permitting the frame to radially expand, spanning the side vessel; and
introducing an index procedure catheter through the access sheath.

2. The method of claim 1, further comprising a control wire extending proximally from the filter and through the sheath, alongside of the index procedure catheter.

3. The method of claim 2, wherein the index procedure comprises a TAVR.

4. The method of claim 1, comprising positioning the distal marker on an upstream side of the brachiocephalic artery.

5. The method of claim 1, comprising positioning the proximal marker on a downstream side of the left subclavian artery.

6. The method of claim 2, further comprising the step of retracting a suture through the control wire to reduce the diameter of the proximal end of the filter.

7. The method of claim 1, further comprising advancing the index procedure catheter through the filter.

8. The method of claim 1, further comprising positioning a distal end of the index procedure catheter distally of the filter.

9. The method of claim 1, wherein the index procedure is a heart valve repair.

10. The method of claim 1, wherein the index procedure is a heart valve replacement.
